(19) United States
(12) Patent Application Publication
(10) Pub. No.: US 2026/0216272 A1
(43) Pub. Date: Jul. 30, 2026

(54) PLECTRANTHUS AMBOINICUS EXTRACT FOR USE IN SCAR COSMESIS

(71) Applicant: ONENESS BIOTECH CO., LTD., Taipei City (TW)

(72) Inventors: Kung-Ming LU, Taipei City (TW); Jui-Ching CHEN, Taipei City (TW)

(21) Appl. No.: 19/152,317

(22) PCT Filed: Feb. 1, 2023
(86) PCT No.: PCT/CN23/74120

§ 371 Date: Jul. 30, 2025

(51) Int. Cl. A61K 36/53; A61K 9/00; A61K 36/23; A61P 17/02; G06T 7/00

(52) U.S. Cl. CPC A61K 36/53; A61K 9/0014; A61K 36/23; A61P 17/02; G06T 7/0014; A61K 2236/33; A61K 2236/35; A61K 2236/51; G06T 2207/30088

(57) ABSTRACT

A method for promoting maturation of a scar or reducing the risk of scar formation in a subject, the method comprising: administering to a subject at a skin site in need thereof an effective amount of a topical formulation, which comprises a Plectranthus amboinicus (PA) extract, optionally in combination with a Centella asiatica (CA) extract, and one or more excipients.

### BACKGROUND OF THE INVENTION

The aesthetics of transverse scars in a perceptible region may impact the appearance as well as elicit functional disorders or pathological morbidities as hypertrophic scars, leading to distress and financial costs to the patients. Therefore, postoperative scar cosmesis is one of the frequently encountered challenges for surgeons. Currently available scar managements include compression therapy, internal agents, surgical resection, intralesional injection, laser therapy, radiotherapy, cryotherapy, and topical drugs. These treatments focus on lessening scar formation after surgeries.

Topical formulations commonly have strong patient compliance owing to their non-invasive, painless and effortlessly administration. Currently available topical anti-scarring agents typically contain conventional silicone-based products, corticosteroids, imiquimod, mitomycin C, onion extraction, etc. and so on. Kim et al., Update on Scar Management: Guidelines for Treating Asian Patients. 2013; 132 (6): 1580-1589. However, the efficacy of such topical formulations is not satisfactory and surgical scar cosmesis remains a challenging task.

### SUMMARY OF THE INVENTION

The present disclosure is based, at least in part, on the development of methods for promoting scar (e.g., post-operative scar) maturation using a topical formulation, as well as the use of a quantitative digital photography assay for assessing scar maturation progress. The topical formulation for use in the methods disclosed herein comprises a Plectranthus amboinicus (PA) extract and optionally a Centella asiatica (CA) extract, as well as excipients such as viscosity increasing agents, emulsifying agents, ointment base, antimicrobial agents, solvents, etc.

Accordingly, the present disclosure features, in some aspects, a method for promoting scar maturation or reducing the risk of scar formation in a subject, the method comprising: administering to a subject at a skin site in need thereof an effective amount of a topical formulation, which comprises a Plectranthus amboinicus (PA) extract, optionally in combination with a Centella asiatica (CA) extract, and one or more excipients. In some embodiments, the scar is a post-operative scar. In some embodiments, the scar is caused by a skin injury (e.g., a trauma scar). In some embodiments, the subject (e.g., a human patient) has undergone a surgical procedure at the skin site (e.g., having scar formed at the surgery site). In other embodiments, the subject (e.g., a human patient) has a surgical procedure scheduled at the skin site.

In some embodiments, the PA extract comprises salvigenin, cirsimaritin, rosmarinic acid, carvacrol, or a combination thereof.

In some embodiments, the PA extract may be prepared by a process comprising: (i) mixing a part of PA (e.g., an above-ground part) with an extracting solution to produce a first PA extract, (ii) filtrating and concentrating the first PA extract to produce a concentrated PA extract; (iii) contacting the concentrated PA extract onto a hydrophobic interaction chromatography resin, and (iv) eluting the column with an eluent solution to product the PA extract. In some instances, the extracting solution is acetone, butyl methyl ether, ethanol, ethyl acetate, isopropyl alcohol, methanol, or a mixture thereof. Alternatively or in addition, the eluent solution comprises a solvent having a polarity index of about 2.1-5.4. In some instances, the eluent solution comprises a mixture of at least two solvents selected from the group consisting of acetone, ethanol, ethyl acetate, and hexane. In some specific examples, the PA extract can be prepared by the process described in Example 1 below.

In some embodiments, the topical formulation disclosed herein may further comprise a CA extract, which may comprise asiaticoside.

In some embodiments, the topical formulation comprises the PA extract and the CA extract at a weight ratio of 1:1 to 1:4. In one example, the weight ratio between the PA extract and the CA extract is 1:4. For example, the topical formulation may comprise about 0.25% (w/w) of the PA extract and/or about 1% (w/w) of the CA extract.

In some embodiments, the topical formulation comprises one or more excipients, which may comprise (a) a viscosity increasing agent, (b) an ointment base, (c) an antimicrobial agent, and (d) an emulsifying agent. In some examples, the viscosity increasing agent in the topical formulation can be about 1.0-10% (w/w). In some examples, the ointment base in the topical formulation can be about 5-30% (w/w). In some examples, the antimicrobial agent in the topical formulation can be about 0.01-0.2% (w/w). In some examples, the emulsifying agent in the topical formulation can be about 0.5-6% (w/w).

Exemplary viscosity increasing agents include, but are not limited to, cetostearyl alcohol, cholesterol, stearyl alcohol, chlorocresol, white wax, stearic acid, cetyl alcohol, or a combination thereof. In one example, the viscosity increasing agent comprises cetostearyl alcohol. Exemplary ointment base includes one or more petrolatum compounds. In one example, the ointment base for use in the topical formulation comprises liquid petrolatum and white petrolatum. Exemplary antimicrobial agents include one or more paraben compounds. In one example, the antimicrobial agent used in the topical formulation comprises methyl paraben and propyl paraben. Exemplary emulsifying agent includes sortiban and/or polysorbate. In one example, the emulsifying agent for use in the topical formulation comprises sorbitan monostearate and polysorbate 60.

In some instances, the one or more excipients in the topical formulation may further comprise one or more solvents, for example, non-water solvent such as propylene glycol, and water.

In specific examples, the topical formulation disclosed herein comprises (e.g., consisting essentially of or consisting of) the Plectranthus amboinicus extract and the Centella asiatica extract in a total amount of about 0.5-5% (w/w), cetostearyl alcohol in an amount of about 1.0-10% (w/w), a combination of white petrolatum and liquid petrolatum in a total amount of about 5-30% (w/w), a combination of methyl paraben and propyl paraben in a total amount of about 0.01-0.2% (w/w), and a combination of sorbitan monostearate and polysorbate 60 in a total amount of about 0.5-6% (w/w).

In some embodiments, the topical formulation is in a form of cream, gel, dressing, spray formulation, ointment, paste, patch, mask, or lotion. In some examples, the topical formulation is in a topical cream formulation. In some embodiments, the topical formulation is administered to the skin site 1-4 times per day (e.g., two times every day).

Any of the methods disclosed herein may further comprise assessing skin conditions at the skin site where the topical formulation is applied before, during, and/or after the treatment. In some embodiments, the skin conditions for assessment may comprise pigmentation, vascularity, pliability, height of scars, hue feature, textual feature, or a combination thereof. In some embodiments, the assessing step comprises semi-quantitative scar assessment, quantitative digital photography analysis, or a combination thereof.

In some embodiments, the assessing step comprises the quantitative digital photography analysis, which comprises: (a) obtaining digital images of one or more scar areas and one or more normal skin areas at the skin site where the topical formulation is applied; (b) processing the digital images to calculate hue features and textural features of the scar areas and the normal skin areas; and (c) comparing the hue features and textual features of the scar areas with those of the normal skin areas to assess scar maturation.

Also within the scope of the present disclosure are topical formulations as those disclosed herein for use in promoting post-operative scar maturation or reducing the risk for post-operative scar formation in a subject, as well as uses of the topical formulation for manufacturing a medicament for use in promoting post-operative scar maturation or reducing the risk for post-operative scar formation in a subject.

In addition, the present disclosure provides a method for assessing scar maturation during a treatment, the method comprising:
(a) obtaining digital images of one or more scar areas and one or more normal skin areas at a skin site where a treatment is applied before and during the course of the treatment;
(b) processing the digital images to calculate hue features and textural features of the scar areas and the normal skin areas; and
(c) comparing the hue features and textual features of the scar areas with those of the normal skin areas to assess scar maturation.

In some embodiments, the treatment comprises a topical formulation, which is applied to a scar site.

The details of one or more embodiments of the invention are set forth in the description below. Other features or advantages of the present invention will be apparent from the following drawings and detailed description of several embodiments, and also from the appended claims.

### BRIEF DESCRIPTION OF THE DRAWINGS

The following drawings form part of the present specification and are included to further demonstrate certain aspects of the present disclosure, which can be better understood by reference to the drawing in combination with the detailed description of specific embodiments presented herein.

FIG. 1 is a schematic diagram showing show a flow chart for quantitative digital photo analysis. Digital photographs of the post-operative scars were taken on day 0 (month 0) as a baseline, and at the 3-month monthly follow-up visits. Photographs of the scars taken were divided into scar and skin sections. The color and texture characteristics of the scars were quantified using image-processing techniques. In addition, difference ratios and relative difference ratios were computed.

FIGS. 2A-2D include representative photos showing post-operative scars at Day 0 (month 0; FIG. 2A), month 1 (FIG. 2B), month 2 (FIG. 2C) and month 3 (FIG. 2D) after treatment by the topical formulation disclosed herein and the placebo control. The left half of the scar (from the patient's position) was treated with topical formulation and the right half was treated with the placebo cream.

FIGS. 3A-3F include diagrams showing trends in the difference ratio and relative difference ratio of the CIELab and RGB systems on the side (left) treated by the topical formulation and side (right) treated by the placebo cream. FIG. 3A: CIELab-L Δ ratio (left panel) and CIELab-L relative Δ ratio (right panel). FIG. 3B: CIELab-a Δ ratio (left panel) and CIELab-a relative Δ ratio (right panel). FIG. 3C: CIELab-b Δ ratio (left panel) and CIELab-b relative Δ ratio (right panel). FIG. 3D: RGB-R Δ ratio (left panel) and RGB-R relative Δ ratio (right panel). FIG. 3E: RGB-G Δ ratio (left panel) and RGB-G relative Δ ratio (right panel). FIG. 3F: RGB-B Δ ratio (left panel) and RGB-B relative Δ ratio (right panel). Statistically significant difference eigenvalues at each month are marked with an asterisk. At month 3, there is a noticeable trend towards an increase in the relative difference ratio on the topical formulation-treated side, which indicates a significant improvement compared to the control side.

FIGS. 4A-4D include graphs showing trends in the difference ratio and relative difference ratio of the texture profiles on the side (left) treated by the topical formulation and the side (right) treated by the placebo cream. FIG. 4A: Contrast Δ ratio (left panel) and Contrast relative Δ ratio (right panel). FIG. 4B: Hom Δ ratio (left panel) and Hom relative Δ ratio (right panel). FIG. 4C: Cor Δ ratio (left panel) and Cor relative Δ ratio (right panel). FIG. 4A: Ent Δ ratio (left panel) and Ent relative Δ ratio (right panel). Statistically significant eigenvalues for each month are marked with an asterisk. Significant trends towards the topical formulation-treated side are displayed.

FIG. 5 includes photos showing improvement of scar maturation after treatment with the topical formulation disclosed herein on a female human patient having post-surgical scar.

### DETAILED DESCRIPTION OF THE INVENTION

Following injury, the skin undergoes a wound healing process culminating in the formation of a mature scar. Wound healing refers to a living organism's replacement of destroyed or damaged tissue by newly produced tissue. The wound healing process involves multiple stages, including inflammatory phase, proliferative phase (with the granulation tissue development and re-epithelialization), and remodeling phase which includes maturation and scar formation (Alhajj et al., StatPearls, 2022).

Many people are left with scars as a result of trauma or surgery. Scar is a nature part of the biological healing process in the skin. During the resolution phase of wound healing, vascular cells and myo-fibroblasts undergo apoptosis, which leads to a large cell number reduction in the granulation tissue (Darby et al., Clin Cosmet Investig Dermatology, 2014). Thereafter, synthesis of ECM is reduced and collagen type III, the major collagen in granulation tissue, is replaced by collagen type I, the dominant skin collagen in normal dermis. Scar tissue is an area of fibrous tissue that develops after an injury to replace normal skin. In some unpredictable situations, the normal scar is developing into pathological fibrotic tissue. It is mainly manifested as fibroblast hyperproliferation, as well as massive deposition of ECM, include collagens, and fibronectin (FN) accounts for the abnormal scarring (Xue et al., Adv Wound Care, 2015). It is also showed reduced tensile strength and elasticity, and a lack of hair follicles.

Pathophysiologic fibrosis, cause by an excessive accumulation of ECM, mainly collagen and fibronectin, results in permanent scar formation (Moretti et al., J Biol Chem, 2022). Mechanical force has been considered as important factors in the development of pathological fibrosis. Fibroblasts become activate, the myofibroblasts, displaying a contractile phenotype in response to mechanical stress (Kubow et al., Nat Commun, 2015). Myofibroblasts also overexpress and secrete transforming growth factor (TGF-β1), α-smooth muscle actin (α-SMA), connective tissue growth factor (CTGF), type I collagen and fibronectin into matrix (Garrett et al., Investig Ophthalmol Vis Sci, 2004).

Myo-fibroblasts, the primary effector cells in scar formation, derive from fibroblasts. In general, aggravated scarring is thought to be associated with c-Jun N-terminal kinase (JNK), TGF-β, Wnty, and Hippo pathways (with enhanced fibroblast migration, increased transition into myofibroblasts, and ECM rearrangement) (Qian et al., Oxidative Med Cell Longev, 2022). In detail, ERK-YAP activation in human cells drive the shift of fibroblasts towards profibrotic phenotypes (Chen et al., Nat Commun, 2021). TGF-β-induced release of IL-11 significantly up-regulate in hypertrophic scars, which activates the enrichment of CD39+ fibroblasts within the upper dermis and secretes a large amount of ECM (Huang et al., J Invest Dermatol, 2022). Keratinocytes secrete the high-mobility group box chromosomal protein 1 (HMGB1) trigger an α-smooth muscle actin promoter by motivating fibroblasts (Zhao et al., J Invest Dermatol, 2018). CXCL4 stimulate myo-fibroblast differentiation and collagen synthesis in fibrotic tissues (Affandi et al., Cell Rep, 2022). N-cadherin has been shown to be critical in injury-triggered swarming, as well as migration of fascia fibroblasts that progressively contract the skin and form scars (Jiang et al., Nat Commun, 2020).

The present disclosure provides topical formulations for use in promoting scar maturation or reducing the risk of scar formation in a subject, for example, a human patient. Also provided herein are quantitative digital photography analysis for use in assessing scar maturation progress in a subject.

### I. Topical Formulations

The instant disclosure herein provides topical formulations for use in promoting scar maturation or reducing the risk of scar formation in a subject. The topical formulation disclosed herein comprise a Plectranthus amboinicus (PA) extract, and suitable excipients and carriers as those disclosed herein. In some embodiments, the topical formulation may further comprise a Centella asiatica (CA) extract.

(a) Plectranthus amboinicus Extract

Plectranthus amboinicus (also known previously or alternatively as Coleus amboinicus Lour., Coleus aromaticus Benth., Coleus aromaticus auct., Plectranthus aromaticus Roxb., Plectranthus aromaticus Benth., and Plectranthus amboinicus (Lour.) Spreng.), is a perennial medicinal herb of the Lamiaceae (also known as Labiatae) family native to Southern and Eastern Africa. Plectranthus amboinicus is also known as patchouli, Cuban oregano, Indian borage, Indian mint, Mexican mint, Mexican oregano, country borage, and Spanish thyme.

A PA extract refers to an extract obtained from a PA plant using one or more suitable solvents. In some examples, the PA extract is prepared using an above-ground part of the PA plant. In some instances, at least one of the solvents used for preparing the extract has a polarity index lower than 7 (e.g., less than 5). See, e.g., U.S. Pat. No. 10,758,584, and International Patent Application No.: PCT/CN2022/101441, the relevant disclosures of each of which are incorporated by reference for the subject matter and purpose referenced herein. As used herein, a solvent refers to a substance or a mixture of substances that dissolves another to form a solution. A PA extract as described herein may be prepared using one solvent. The solvent used in each extracting step for preparing the extracts described herein (including both PA and CA extracts) may be a single solvent. Alternatively, it can be a mixture of two or more solvents.

The PA extract described herein may comprise terpenoids (e.g., monoterpenoids, diterpenoids, triterpenoids, and/or sesquiterpenoids), flavonoids, phenolics, essential oils, or a combination thereof. The PA extract for making the pharmaceutical compositions disclosed herein may comprise salvigenin, and optionally one or more of cirsimaritin, rosmarinic acid, and carvacrol.

The PA extract described herein may be prepared by extracting a whole PA plant or a part thereof (e.g., an above-ground part) with one or more suitable solvents to produce a solution and then drying the solution to produce the PA extract. Since the PA extract comprises flavonoids, terpenoids (e.g., monoterpenoids, diterpenoids, triterpenoids, and/or sesquiterpenoids), phenolics, or essential oils, which are non-polar molecules, at least one of the extracting solvents may have a relatively low polarity (e.g., having a polarity index lower than 7) to facilitate dissolution of the non-polar molecules. “Extracting” can be performed by either contacting the PA material directly with a suitable solvent or by eluting active components of PA from resins on which the active components are attached.

In some examples, a solvent having a polarity index lower than 7 can be used for extracting the active components from PA to produce the PA extract. Such a solvent can be ethyl acetate, methyl acetate, propanol, butanol, or chloroform. Alternatively, the solvent can be a mixture of one or more solvents having different polarity index. Examples include, but are not limited to, a mixture of ethanol and ethyl acetate, ethyl acetate and butanol, ethanol and propanol, methyl acetate and butanol.

In some embodiments, the PA extract may be prepared by a process involving the use of one solvent, such as a solvent having a polarity index lower than 7 (e.g., <about 6.5, <about 6.0; <about 5.5, <about 5.0, <4.9, <4.8, <4.7, <4.6, or <4.5). Examples include, but are not limited to, methanol, ethanol, acetone, ethyl acetate, butanol, dichloromethane, or a combination thereof.

PA materials, which can be a whole PA or a part thereof (e.g., an above-ground part such as leaves), can be prepared by routine methodology. The PA material can be a fresh plant or a part thereof. Alternatively, the PA material can be in dried form. The PA can optionally be dried to form powders, which can be used as the PA material for preparing the PA extract.

Any of the PA materials as described herein can be extracted, one or more times, by a suitable solvent to produce a crude extract. The solvent for use in preparing the crude extract may be a high-polarity solvent, for example, having a polarity index above 5 and preferably below 7 (e.g., >5.2; >5.5, >5.8, >6 or above and preferably below 7). Examples include, but are not limited to, ethanol, acetone, methanol, water, or a combination thereof. If necessary, the crude extract can be concentrated by a conventional method to produce a concentrated crude extract.

The crude extract can then be brought in contact with a suitable resin (e.g., a non-ionic absorbent resin) under suitable conditions that allow for binding of active components in the crude extract onto the resin. Exemplary resins for use in preparing the Plectranthus amboinicus extract include, but are not limited to, DIAION® HP20, DIAION® HP20SS, Sepabeads® SP207, Amberlite™ XAD-2, or Amberlite™ XAD-4.

Afterwards, the resin can be washed one or more times and eluted with a suitable solvent, for example, a solvent having a polarity index lower than 7, to produce a PA extract, which can then be dried by a conventional method (e.g., freeze-drying, spray-drying, or concentration drying) to produce dried the PA extract, which can be in semisolid or paste form.

In some examples, the resin absorption step can be performed by mixing the crude extract with the resin in a container. In other embodiments, the resin separation step can be performed by a chromatography column setting.

In one example, an extract of PA can be prepared as follows. An overground part of PA (about 1.5 g), including leaves and/or stems, can be collected and extracted with a solvent having a polarity less than 7 (e.g., methanol, ethanol, acetone, ethyl acetate, butanol, dichloromethane, or a combination thereof) at room temperature for 30 min to 6 hours. Alternatively, this extraction process can be carried out at a temperature of about 50 to 80° C. The resultant crude extract can be directly loaded to a non-ionic adsorbent resin column and eluted by a solvent having a polarity less than 6 (e.g., ethanol, ethyl acetate, butanol, dichloromethane, hexane, toluene, or a combination thereof). The eluted components can be collected and purified by extraction with a solvent having a polarity less than 6 (e.g., those described herein). The resultant filtrate can be collected to produce a PA extract.

One specific example of the PA preparation process is provided in Example 1. In some instances, the PA extract used in any of the topical formulations disclosed herein may be prepared by the process described in Example 1.

(b) Centella asiatica Extract

Centella asiatica (also known previously or alternatively as Centella asiatica Urban, Centella asiatica (L.) Urban, Hydrocotyle asiatica L., and Trisanthus cochinchinensis Lour.) is a perennial medicinal plant of the family Mackinlayaceae or subfamily Mackinlayoideae of the Apiaceae (also known as Umbelliferae) family native to Asia, Africa, and South America. Centella asiatica is also known as European water-marvel, gotu kola, Kola, pennywort, Indian pennywort, marsh pennywort, pennyweed, Indian ginseng, Horse-hoof grass, Pegaga, Mandookaparni, Tiger herbal, Spadeleaf, or Tono. Extracts of Centella asiatica generally comprise two major compounds: asiaticoside and madecassic acid.

In some embodiments, the topical formulation disclosed herein may further comprise a CA extract, which may comprise the asiaticoside compound. The CA extract as described herein refers to an extract obtained from whole CA plants or a part thereof. The CA extract may comprise asiaticoside, and optionally madecassic acid.

The CA extract can be prepared following a conventional method, for example, those described in U.S. Pat. Nos. 5,834,437, 6,417,349, 6,475,536, and 6,267,996, CN 1313124, CN 1089497, and CN 1194154. Below is an example.

CA materials can be prepared via routine practice. Such materials can be fresh CA plant or a part thereof, or dried CA. The CA materials can be extracted with a suitable solvent such as water, ethanol, or a mixture thereof, to produce a crude extract. The crude extract, which can be optionally concentrated, can be either mixed with a suitable resin or loaded onto a column packed with the resin. After being washed for one or more times, the resin can be eluted with a suitable solvent. The resultant eluent can be concentrated to form a paste, which can be dried by a conventional method, for example, vacuum dry, to produce powders of the CA extract. When necessary, the CA powder can be grinded through a mesh (e.g., a No. 100 mesh).

An extract of CA can be prepared by the same or similar process as described above for making the PA extract. The PA Extract and/or CA Extract may be concentrated using a pressure-reducing rotary evaporator.

### (c) Suitable Excipients and Carriers

In some instances, the topical formulation may further comprise one or more suitable carriers or excipients, for example, one or more of viscosity increasing agents (e.g., about 1.0-10%), one or more ointment bases (e.g., one or more cream base) which may range from about 5-30%, one or more antimicrobial preservative (e.g., about 0.005-0.2% such as about 0.01-0.2% by weight), one or more emulsifying agents (about 0.5-10% such as about 0.5-6% by weight) or a combination thereof. These components may be dissolved or disbursed in a suitable solvent.

As used herein, the term “about” intends to a strict numerical boundary to the specified parameters (including both the upper and lower limits). A skilled person in the art would have understood the meaning of “about” in association with a specific context. In some instances, the term “about” refers to a particular value +/−5% (e.g., +/−3% or +/−2%).

A “viscosity increasing agent” is an agent that is used to thicken a formulation. Exemplary viscosity increasing agents may include, for example, cetostearyl alcohol, cholesterol, stearyl alcohol, chlorocresol, white wax, stearic acid, cetyl alcohol, or a combination thereof. The viscosity increasing agent may be present in the topical formation at a concentration of about 1.0-10% (w/w). For example, the topical formulation may comprise about 1-1.5%, 1.5-2%, 2-2.5%, 2.5-3%, 3-3.5%, 3.5-4%, 4-4.5%, 4.5-5%, 5-5.5%, 5.5-6%, 6-6.5%, 6.5-7%, 7-7.5%, 7.5-8%, 8-8.5%, 8.5-9%, 9-9.5%, or 9.5-10% (w/w) of the viscosity increasing agent. Alternatively, the topical formulation may comprise about 1-5%, 2.5-7.5%, or 5-10% (w/w) of the viscosity increasing agent. In specific examples, the topical formulation may comprise cetostearyl alcohol as the emulsifying agent, which may be in an amount of about 4-6% (w/w) in the topical formulation.

An “ointment base” can be any semisolid preparation or vehicle into which an active agent may be incorporated. Exemplary ointment bases include, but are not limited to, oleaginous ointment bases (e.g., white petrolatum or white ointment), absorption ointment bases (e.g., hydrophilic petrolatum, anhydrous lanolin, Aquabase™, Aquaphor®, and Polysorb®), water/oil emulsion ointment bases (e.g., cold cream, hydrous lanolin, rose water ointment, Hydrocream™, Eucerin®, and Nivea®), oil/water emulsion ointment bases (e.g., hydrophilic ointments, Dermabase™, Velvachol®, and Unibase®), and water-miscible ointment bases (e.g., polyethylene glycol (PEG) ointment and Polybase™). Ointment bases may be pharmacologically inert but can entrap water in order to provide an emollient protective film. In a specific embodiment, the ointment base may be any petrolatum compound (e.g., petrolatum, white petrolatum, white soft paraffin, liquid petrolatum, liquid paraffin). In a further specific embodiment, the ointment base comprises white petrolatum (e.g., CAS number 8009-03-8). In some examples, the ointment base for use in the topical formulation disclosed herein contains a combination of liquid petrolatum and white petrolatum. The ointment base may be present in the topical formation at a concentration of about 5-30% (w/w), e.g., 10-30% (w/w). For example, the topical formulation may comprise about 5-25%, 5-20%, 5-15%, 5-15%, 10-15%, 15-20%, 20-25%, or 25-30% (w/w) of the ointment base. Specifically, the topical formulation may comprise about 5, 6, 7, 8, 9, 10, 11, 12, 13, 14, 15, 16, 17, 18, 19, 20, 21, 22, 23, 24, 25, 26, 27, 28, 29, or 30 percent (w/w) of the ointment base. In specific examples, the topical formulation may contain about 20-30% (w/w) of the combination of liquid petrolatum and white petrolatum as the ointment base.

In some embodiments, the “ointment base” described herein contains less than 20% water and volatiles, and more than 50% hydrocarbons, waxes, or polyols as the vehicle.

In some embodiments, the “ointment base” described herein is a “cream base,” which contains more than 20% water and volatiles and/or typically contain less than 50% hydrocarbons, waxes, or polyols as the vehicle for the drug substance. The cream base can be a multiphase preparation containing a lipophilic phase and an aqueous phase. In some instances, the cream base is a lipophilic cream base, which has a lipophilic phase as the continuous phase. Such a cream base usually contains water-in-oil emulsifying agents such as wool alcohols, sorbitan esters and monoglycerides. In other instances, the cream base is a hydrophilic cream base, which has an aqueous phase as the continuous phase. Such a cream base typically contains oil-in-water emulsifying agents such as sodium or trolamine soaps, sulfated fatty alcohols, polysorbates and polyoxyl fatty acid and fatty alcohol esters, which may be in combination with water-in-oil emulsifying agents, if needed.

An “antimicrobial preservative” can be any compound capable of destroying microbes, prevent the multiplication or growth of microbes, or prevent the pathogenic action of microbes. Exemplary antimicrobial preservatives include, but are not limited to, a paraben compound (an ester of para-hydroxybenzoic acid; e.g., paraben, methylparaben, ethylparaben, propylparaben, butylparaben, heptylparaben, benzylparaben, isobutylparaben, isopropylparaben, benzylparaben, or their sodium salts), benzalkonium chloride, benzethonium chloride, benzyl alcohol, boric acid, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and thimerosal. The antimicrobial preservative may be present in the topical formation at a concentration of about 0.005-0.2%, e.g., about 0.01-0.2% (w/w). For example, the topical formulation may comprise about 0.005-0.01%, 0.01-0.05%, 0.05-0.1%, 0.1-0.15%, or 0.15-0.2% (w/w) of the antimicrobial preservative. Specifically, the topical formulation may comprise about 0.005, 0.006, 0.007, 0.008, 0.009, 0.01, 0.02, 0.03, 0.04, 0.05, 0.06, 0.07, 0.08, 0.09, 0.1, 0.11, 0.12, 0.13, 0.14, 0.15, 0.16, 0.17, 0.18, 0.19, or 0.2 percent (w/w) of the antimicrobial preservative. In some examples, the topical formulation contains about 0.08-0.15% (w/w) of methyl paraben and propyl paraben in combination.

An “emulsifying agent” is a compound or substance which acts as a stabilizer for a mixture of two or more liquids that are normally immiscible (unmixable or unblendable). Exemplary emulsifying agents may include, but are not limited to, natural emulsifying agents (e.g., acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g., bentonite [aluminum silicate] and Veegum [magnesium aluminum silicate]), long chain amino acid derivatives, high molecular weight alcohols (e.g., stearyl alcohol, cetyl alcohol, oleyl alcohol, triacetin monostearate, ethylene glycol distearate, glyceryl monostearate, propylene glycol monostearate, and polyvinyl alcohol), carbomers (e.g., carboxy polymethylene, polyacrylic acid, acrylic acid polymer, and carboxyvinyl polymer), carrageenan, cellulosic derivatives (e.g., carboxymethylcellulose sodium, powdered cellulose, hydroxymethyl cellulose, hydroxypropyl cellulose, hydroxypropyl methylcellulose, and methylcellulose), sorbitan fatty acid esters (e.g., polyoxyethylene sorbitan monolaurate [Tween® 20], polyoxyethylene sorbitan [Tween® 60], polyoxyethylene sorbitan monooleate [Tween® 80], sorbitan monopalmitate [Span® 40], sorbitan monostearate [Span® 60], sorbitan tristearate [Span® 65], glyceryl monooleate, and sorbitan monooleate [Span® 80]), polyoxyethylene esters (e.g., polyoxyethylene monostearate [Myrj® 45], polyoxyethylene hydrogenated castor oil, polyethoxylated castor oil, polyoxymethylene stearate, and Solutol), sucrose fatty acid esters, polyethylene glycol fatty acid esters (e.g., Cremophor®), polyoxyethylene ethers (e.g., polyoxyethylene lauryl ether [Brij® 30]), and poly(vinyl-pyrrolidone), diethylene glycol monolaurate, triethanolamine oleate, sodium oleate, potassium oleate, ethyl oleate, oleic acid, ethyl laurate, sodium lauryl sulfate, Pluronic F 68, Poloxamer 188, cetrimonium bromide, cetylpyridinium chloride, benzalkonium chloride, and docusate sodium, and/or combinations thereof. The emulsifying agent may be present in the topical formation at a concentration of about 0.5-10% (w/w), e.g., 0.5-6% (w/w). For example, the topical formulation may comprise about 0.5-1%, 1-1.5%, 1.5-2%, 2-2.5%, 2.5-3%, 3-3.5%, 3.5-4%, 4-4.5%, 4.5-5%, 5-5.5%, 5.5-6%, 5-10%, 6-10%, or 8-10% (w/w) of the emulsifying agent. Specifically, the topical formulation may comprise about 0.5, 0.6, 0.7, 0.8, 0.9, 1, 1.5, 2, 2.5, 3, 3.5, 4, 4.5, 5, 5.5, 6, 6.5, 7, 7.5, 8, 8.5, 9, 9.5, or 10 percent (w/w) of the emulsifying agent. In some examples, the topical formulation may comprise about 2-4% (w/w) of sorbitan monostearate and polysorbate 60 as the emulsifying agent.

The topical formulation of the invention may further contain one or more solvents (e.g., non-water solvents or water). Exemplary non-water solvents may include, but are not limited to, any known solvent including propylene glycol, glycol, and mixtures thereof. The non-water solvent may be present in the topical formation at a concentration of about 2-65% (w/w). For example, the topical formulation may comprise about 2-15%, 15-30%, 30-45%, or 45-65% (w/w) of the solvent. In some embodiments, the topical formulation as disclosed herein may also contain water.

In some embodiments, the topical formulation of the invention may further comprise one or more emollients, fragrances, or pigments. The topical formula may also be used in conjunction with a wound dressing (e.g., bandage with adhesive, plaster patch and the like). (e.g., cyclohexane, n-hexane, n-decane, i-octane, octane, butyl ether, carbon tetrachloride, triethyl amine, i-propyl ether, toluene, p-xylene, t-butyl methyl ether, benzene, benzyl ether, dichloromethane, methylene chloride, chloroform, dichloroethane, ethylene dichloride, 1-butanol, i-butyl alcohol, tetrahydrofuran, ethyl acetate, 1-propanol, 2-propanol, methyl acetate, cyclohexanone, methyl ethyl ketone (MEK), nitrobenzene, benzonitrile, 1,4-dioxane, or p-dioxane).

Additional information regarding topical formulations can be found, e.g., in U.S. Pat. No. 10,758,584, the relevant disclosures of which are incorporated by reference for the subject matter and purpose referenced herein.

The topical formulation disclosed herein may be in any suitable format. Examples include, but are not limited to, cream, dressing, ointment, lotion, paste, mask, and gel. In some instances, the topical formulation may be placed on a device (e.g., a patch) for applying to a site where skin disorders occur.

### II. Methods for Promoting Scar Maturation

Any of the topical formulations disclosed herein can be used for promoting scar maturation or reducing the risk of scar formation in a subject such as in a human subject. To practice the method disclosed herein, an effective amount of the topical formulation can be administered to a subject (e.g., a human) in need of the treatment via a suitable route, such as topical application to a skin site where scar forms or to be formed (e.g., a surgery site or a site where a surgical procedure is to be performed).

In some instances, a topical formulation as described herein can be used for promoting maturation of a post-operative scar or reducing the risk of post-operative scar formation. In some instances, the topical formulation disclosed herein can be used to promote maturation of a scar caused by trauma. In some examples, the topical formulation can be used to promote maturation of a hypertrophic scar. A hypertrophic scar (HTS) is a thickened, wide, raised scar and is still a significant challenge in wound management after injury. Hypertrophic scars are a fibro-proliferative disorder and considered a defect in myo-fibroblast apoptosis could be responsible for the pathological scar formation, since myo-fibroblast overproduces collagen, which causes the scar to be raised above the surrounding skin (Moulin et al., J Cell Physiol, 2004).

The topical formulation may be applied to a scar site or a site that is at risk for scar formation following a suitable dosage and treatment regimen. The dosage and administration regimen for the described method will depend on the nature and condition of the symptoms being treated, the age and condition of the patient, and any prior or concurrent therapy. In some instances, the topical formulation can be applied once every week, once every other day, once daily, twice daily, three times daily, or four time daily for a suitable period of time.

The subject to be treated by the topical formulation disclosed herein can be a human or a non-human mammal. In some embodiments, the subject is a human patient having a scar (e.g., a post-operative scar, a skin injury scar, a keloid scar, or a hypertrophic scar) or is at risk for scar formation (e.g., will be subject to a surgery that can cause scar formation post-surgery). A subject having a scar as those disclosed herein can be identified by routine medical examinations.

As used herein, “an effective amount” refers to the amount of each active agent required to confer therapeutic effect on the subject, either alone or in combination with one or more other active agents. Determination of whether an amount of the composition achieved the therapeutic effect would be evident to one of skill in the art. Effective amounts vary, as recognized by those skilled in the art, depending on the particular condition being treated, the severity of the condition, the individual patient parameters including age, physical condition, size, gender and weight, the duration of the treatment, the nature of concurrent therapy (if any), the specific route of administration and like factors within the knowledge and expertise of the health practitioner. These factors are well known to those of ordinary skill in the art and can be addressed with no more than routine experimentation. It is generally preferred that a maximum dose of the individual components or combinations thereof be used, that is, the highest safe dose according to sound medical judgment.

Empirical considerations, such as the half-life, generally will contribute to the determination of the dosage. Frequency of administration may be determined and adjusted over the course of therapy, and is generally, but not necessarily, based on treatment and/or suppression and/or amelioration and/or delay of a target disease/disorder. Alternatively, sustained continuous release formulations of the composition may be appropriate. Various formulations and devices for achieving sustained release are known in the art.

In one example, dosages for a composition as described herein may be determined empirically in individuals who have been given one or more administration(s) of the composition. Individuals are given incremental dosages of the agonist. To assess efficacy of the agonist, an indicator of the disease/disorder can be followed.

Generally, for administration of any of the compositions described herein, an initial candidate dosage can be given to a subject. For repeated administrations over several days or longer, depending on the condition, the treatment is sustained until a desired suppression of symptoms occurs or until sufficient therapeutic levels are achieved to alleviate a target disease or disorder, or a symptom thereof. The particular dosage regimen, i.e., dose, timing and repetition, will depend on the particular individual and that individual's medical history, as well as the properties of the individual agents (such as the half-life of the agent, and other considerations well known in the art).

For the purpose of the present disclosure, the appropriate dosage of the composition as described herein will depend on the specific active agent contained therein, the type and severity of the disease/disorder, whether the composition is administered for preventive or therapeutic purposes, previous therapy, the patient's clinical history and response to treatment, and the discretion of the attending physician. Typically, the clinician will administer the composition, until a dosage is reached that achieves the desired result. In some embodiments, the desired result is a reduction of disease severity (e.g., represented by a disease score). Methods of determining whether a dosage resulted in the desired result would be evident to one of skill in the art. Administration of any of the compositions disclosed herein can be continuous or intermittent, depending, for example, upon the recipient's physiological condition, whether the purpose of the administration is therapeutic or prophylactic, and other factors known to skilled practitioners. The administration of the composition may be essentially continuous over a preselected period of time or may be in a series of spaced dose, e.g., either before, during, or after developing a target disease or disorder.

As used herein, the term “treating” refers to the application or administration of a composition including one or more active agents to a subject, who has a target disease or disorder, a symptom of the disease/disorder, or a predisposition toward the disease/disorder, with the purpose to cure, heal, alleviate, relieve, alter, remedy, ameliorate, improve, or affect the disorder, the symptom of the disease, or the predisposition toward the disease or disorder.

Alleviating a target disease/disorder includes delaying the development or progression of the disease or reducing disease severity or prolonging survival. Alleviating the disease or prolonging survival does not necessarily require curative results. As used therein, “delaying” the development of a target disease or disorder means to defer, hinder, slow, retard, stabilize, and/or postpone progression of the disease. This delay can be of varying lengths of time, depending on the history of the disease and/or individuals being treated. A method that “delays” or alleviates the development of a disease, or delays the onset of the disease, is a method that reduces probability of developing one or more symptoms of the disease in a given time frame and/or reduces extent of the symptoms in a given time frame, when compared to not using the method. Such comparisons are typically based on clinical studies, using a number of subjects sufficient to give a statistically significant result.

“Development” or “progression” of a disease means initial manifestations and/or ensuing progression of the disease. Development of the disease can be detectable and assessed using standard clinical techniques as well known in the art. However, development also refers to progression that may be undetectable. For purpose of this disclosure, development or progression refers to the biological course of the symptoms. “Development” includes occurrence, recurrence, and onset. As used herein “onset” or “occurrence” of a target disease or disorder includes initial onset and/or recurrence.

The particular dosage regimen, i.e., dose, timing and repetition, used in the method described herein will depend on the particular subject and that subject's medical history.

In some embodiments, any of the compositions disclosed herein may be co-used with one or more additional therapeutic agents for treating the target disease. In some instances, the additional therapeutic agents may serve to enhance and/or complement the effectiveness of the composition disclosed herein.

Treatment efficacy for a target disease/disorder can be assessed by methods well-known in the art.

### III. Assessment of Scar Maturation

In some aspects, provided herein are assessment methods for monitoring scar maturation in a subject, who may be subject to a treatment to improve scar maturation (e.g., involving any of the topical formulation as disclosed herein). For example, the assessment analysis may be performed before, during the course, and/or after treatment with the topical formulation to monitor efficacy and scar maturation progress in a subject. The assessment may involve evaluation of skin conditions at the scar site and optionally at nearby normal skin sites over the course of the treatment. Such skin conditions may comprise pigmentation, vascularity, pliability, height of scars, hue (color) feature, textual feature, or a combination thereof.

In some embodiments, the assessment method may comprise comprises semi-quantitative scar assessment, quantitative digital photography analysis, or a combination thereof. The semi-quantitative scar assessment assays may comprise the Vancouver Scar Scale (VSS) and/or modified Vancouver Scar Scale (mVSS) approaches as known in the art. See also Example 2 below.

In some instances, the assessment method disclosed herein comprises the quantitative digital photography analysis, which involves obtaining digital images of skin sites and processing such digital images to obtain changes of skin features (e.g., color features and/or textual features) over the course of treatment. Comparison of such features between scar site and normal skin site may be involved to provide further information for treatment efficacy/scar maturation progress. The color of scarring is deemed to be associated with scar maturation, and CEILab and Hue are colorimetric values for quantification and communication (Draaijers et al., Burns, 2004). The texture can be divided into color, size, and shape, which generates perception of density, coarseness, fineness, smoothness (Kwak et al., Expert Syst Appl, 2015).

In some instances, the digital photography analysis disclosed herein may comprise analysis of contrast, correlation, homogeneity, and/or entropy of the texture information.

In some examples, the digital photography analysis may comprise: (a) obtaining digital images of one or more scar areas and one or more normal skin areas at a skin site in need thereof (e.g., where a treatment such as the topical formulation is applied); (b) processing the digital images to calculate hue features and textural features of the scar areas and the normal skin areas; and (c) comparing the hue (color) features and textual features of the scar areas with those of the normal skin areas to assess scar maturation.

### IV. Kits for Use in Promoting Scar Maturation

The present disclosure also provides kits for use in promoting scar maturation in a subject who needs the treatment. Such kits may include one or more containers comprising any of the topical formulations as described herein, which comprises the Plectranthus amboinicus extract, optionally in combination with the Centella asiatica extract, or one or more active agents thereof such as those disclosed herein, and one or more suitable excipients and carriers as also disclosed herein.

In some embodiments, the kit may comprise instructions for use in accordance with any of the methods described herein. The included instructions may comprise a description of administration of the topical formulation to promote scar maturation or to reduce the risk of scar formation according to any of the methods described herein. The kit may further comprise a description of selecting an individual suitable for treatment based on identifying whether that individual has scar or is at risk for scar formation.

The instructions relating to the use of the topical formulation generally include information as to dosage, dosing schedule, and route of administration for the intended treatment. The containers may be unit doses, bulk packages (e.g., multi-dose packages) or sub-unit doses. Instructions supplied in the kits of the invention are typically written instructions on a label or package insert (e.g., a paper sheet included in the kit), but machine-readable instructions (e.g., instructions carried on a magnetic or optical storage disk) are also acceptable.

The label or package insert indicates that the topical formulation is used for promoting scar maturation. Instructions may be provided for practicing any of the methods described herein.

The kits of this invention are in suitable packaging. Suitable packaging includes, but is not limited to, vials, bottles, jars, flexible packaging (e.g., sealed Mylar or plastic bags), and the like. Kits may optionally provide additional components such as interpretive information. Normally, the kit comprises a container and a label or package insert(s) on or associated with the container. In some embodiments, the invention provides articles of manufacture comprising contents of the kits described above.

Without further elaboration, it is believed that one skilled in the art can, based on the above description, utilize the present invention to its fullest extent. The following specific embodiments are, therefore, to be construed as merely illustrative, and not limitative of the remainder of the disclosure in any way whatsoever. All publications cited herein are incorporated by reference for the purposes or subject matter referenced herein.

### General Techniques

The practice of the present disclosure will employ, unless otherwise indicated, conventional techniques of molecular biology (including recombinant techniques), microbiology, cell biology, biochemistry, and immunology, which are within the skill of the art. Such techniques are explained fully in the literature, such as Molecular Cloning: A Laboratory Manual, second edition (Sambrook, et al., 1989) Cold Spring Harbor Press; Oligonucleotide Synthesis (M. J. Gait, ed. 1984); Methods in Molecular Biology, Humana Press; Cell Biology: A Laboratory Notebook (J. E. Cellis, ed., 1989) Academic Press; Animal Cell Culture (R. I. Freshney, ed. 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds. 1993-8) J. Wiley and Sons; Methods in Enzymology (Academic Press, Inc.); Handbook of Experimental Immunology (D. M. Weir and C. C. Blackwell, eds.): Gene Transfer Vectors for Mammalian Cells (J. M. Miller and M. P. Calos, eds., 1987); Current Protocols in Molecular Biology (F. M. Ausubel, et al. eds. 1987); PCR: The Polymerase Chain Reaction, (Mullis, et al., eds. 1994); Current Protocols in Immunology (J. E. Coligan et al., eds., 1991); Short Protocols in Molecular Biology (Wiley and Sons, 1999); Immunobiology (C. A. Janeway and P. Travers, 1997); Antibodies (P. Finch, 1997); Antibodies: a practice approach (D. Catty., ed., IRL Press, 1988-1989); Monoclonal antibodies: a practical approach (P. Shepherd and C. Dean, eds., Oxford University Press, 2000); Using antibodies: a laboratory manual (E. Harlow and D. Lane (Cold Spring Harbor Laboratory Press, 1999); The Antibodies (M. Zanetti and J. D. Capra, eds. Harwood Academic Publishers, 1995); DNA Cloning: A practical Approach, Volumes I and II (D. N. Glover ed. 1985); Nucleic Acid Hybridization (B. D. Hames & S. J. Higgins eds. (1985»; Transcription and Translation (B. D. Hames & S. J. Higgins, eds. (1984»; Animal Cell Culture (R. I. Freshney, ed. (1986»; Immobilized Cells and Enzymes (IRL Press, (1986»; and B. Perbal, A practical Guide To Molecular Cloning (1984); F. M. Ausubel et al. (eds.).

Without further elaboration, it is believed that one skilled in the art can, based on the above description, utilize the present invention to its fullest extent. The following specific embodiments are, therefore, to be construed as merely illustrative, and not limitative of the remainder of the disclosure in any way whatsoever. All publications cited herein are incorporated by reference for the purposes or subject matter referenced herein.

### Example 1: Exemplary Plectranthus amboinicus Extract Preparation Process

For extraction, 1 kg of the above ground part of Plectranthus amboinicus was stirred in 10-20 L ethanol at 60±5° C. for 90 minutes. This step was repeated twice and the resultant mixture was filtered and concentrated using rotary evaporation until the volume of the concentrated extract was about 30-70% of the original volume. Without dilution, the concentrated solution was loaded onto a column packed with DIAION® HP-20 resin (a non-polar copolymer styrene-divynilbenzene adsorbent resin). The column was eluted with an eluent solution containing ethanol and ethyl acetate at a volume ratio of about 1:1. The eluted fraction was collected and dried to produce a PA extract (yield: 1.2%).

### Example 2. Validation of Topical Formulation Comprising Herb Extracts in Postoperative Scar Cosmesis Using Quantitative Digital Photography Analysis

Unsightly scarring after surgery remains a problem. This study aims to evaluate the efficacy of a topical formulation comprising a combination of a Plectranthus amboinicus (PA) extract and a Centella asiatica (CA) extract for preventing the formation of and/or alleviating post-skin incision scars. This study also aims at developing a digital analysis system as a sensitive tool to assess scar improvement.

This study is a prospective, placebo-controlled trial involved patients with post-operative transverse scars. Each patient received a topical formulation on the left scar (from patient's position), and a placebo cream on the right scar (from patient's position). In addition to subjective VSS, mVSS and VAS scores, a digital photography was utilized for scar analysis monthly with colorimetric analysis, such as CIELab and hue, and contrast, correlation, homogeneity, entropy as texture information.

46 patients, with a mean age 52, participated in this study. All the parameters of subjective assessment showed superior results on the topical formulation-treated side with significant differences in pigmentation, vascularity, pliability, height, itchiness and patient's satisfaction (p=0.043, 0.013, 0.026, 0.002, 0.039, 0.012, respectively). The trend of color and texture showed increased relative difference ratio with significant difference in most of the eigenvalues toward the topical formulation treated side, including CIELab-L (p<0.0001), Hue-R,B,G (p=0.034, 0.001, 0.011), contrast (p<0.0001), homogeneity (p<0.0001), correlation (p=0.011), and entropy (p<0.0001). The results show superior efficacy of the topical formulation in promoting post-operative scar maturation from not only subjective assessments but also with objective quantitative analyses. The results also indicated that the digital photographic quantitative analysis system is an ideal tool for quantification of scar appearance.

### Method

### Study Design

This study was designed as a prospective, double-blind, placebo-controlled trial involving 46 patients with transverse scars to explore the relationship between the herb extract-containing topical formulation and post-surgical scarring based on subjective and objective assessment. Bound by the midpoint of the scar, each patient received the topical formulation (in cream format) on the left scar, and a placebo cream on the right scar. Treatment was given twice a day for 84 days. Assessments of the scars were performed and photographed clinically on a monthly basis for 3 months, at Day 0, Day 28, Day 56, and Day 84, following the first topical application using three methods: a clinical assessment using the Vancouver Scar Scale (VSS) and modified Vancouver Scar Scale (mVSS), a quantitative digital photography analysis established before and after treatment improvements, and at the end of the study period, patients completed a final satisfaction questionnaire in combination with a Visual Analogue Scale (VAS).

### Inclusion and Exclusion Criteria

Eligible subjects had transverse scarring in their neck or abdomen following elective surgery, including thyroid and parathyroid surgery, excision of a neck mass, inguinal hernia repair, or any neck or abdominal surgeries which can be managed by primary closure of clean surgical wounds.

The exclusion criteria included previous trauma or radiation therapy that would affect the surgical incision site, medical history with chemo-therapy or targeted therapy for any reason, pregnancy, abnormal laboratory values at screening as anemia, sepsis, autoimmune disease, liver function studies over 3 times of the upper limit of normal, renal function studies over 2 times of the upper limit of normal, chronic alcohol or drug abuse problem, or the presence of any clinically significant condition that could pose a threat to subject compliance or interfere with wound healing.

A total of 46 patients of either sex, aged 20-92 years, were enrolled in this study.

### Preparation of Topical Cream Formulation

The topical formulation comprises 1.25% (w/w) herb extracts, including a Plectranthus amboinicus exact (0.25%) and a Centella asiatica extract (1%). The placebo cream contains the same excipients and carriers as the topical formulation but not the PA and CA extracts. Both samples were prepared to be identical in terms of consistency and appearance in yellow-green to light green color and placed in the same dispensers but labeled left and right respectively. The topical cream or the placebo cream was applied twice daily to the transverse scar areas of a patient for up to 3 months (84 days).

### Assessment of Scars

(a) Subjective Scoring for Scar Quality with Semi-Quantitative Assessments

At the end of the study period (month 3, day 84), three independent observers including an otolaryngologist, a gynecologist, and a plastic surgeon made objective clinical assessments of both left and right side of the scars by the VSS, which was developed in 1990 by Sullivan et al., delineated for clinician assessments by four parameters including pigmentation, vascularity, pliability and height of scars (Sullivan et al., J Burn Care Rehab, 1990). In addition, the principal investigator utilized the mVSS by Nedelec et al. to assess scars with additional indicators of subjective symptoms, such as pain and itchiness, and modified scale for pigmentation (In generally, the higher VSS and mVSS score represents the uglier scar formation) (Nedelec et al., J Burn Care Rehab, 2000).

Moreover, the patients completed a final satisfaction questionnaire, using VAS, a numeric rating scale from 0 to 10, to quantify their satisfaction of scar cosmesis and the impact of scar appearance to their quality of life (QoL).

### (b) Quantitative Digital Photography Analysis Processing

### Image Processing

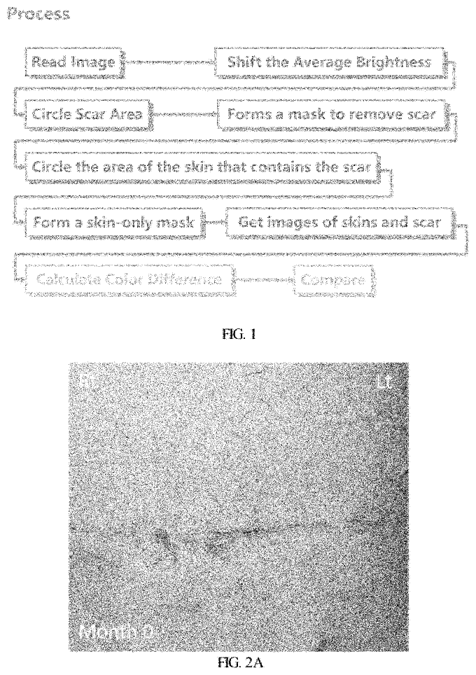

184 standard anterior/posterior view photographs of scars, including 4 images for each patient, before and after the treatment for 3 months were taken. A Canon EOS 850D was employed for all photographs, with image quality set to the highest level possible to catch the details of scars. Distance from subject to lens barrel was standardized to 30 cm. The digital photography was captured and processed by MATLAB. FIG. 1 depicts an exemplary image processing pathway for quantifying the scar variation with color and texture features used in this study. These photographs of scars were captured and divided into scar parts and skin parts nearby the scar site. The difference between the scar part and the nearby skin part was calculated and expressed as delta (4), and the photographs taken on day 0 (month 0, M0) as baseline. Meanwhile, the difference ratio and relative difference ratio were calculated. Difference ratio represents difference between scar and skin out of skin (Δ ratio=Δ(scar−skin)/skin). Using the data of M0 as baseline, relative Δ ratio represents the difference of A ratio between Mx and M0 out of baseline (relative Δ ratio=(Mx Δ ratio−M0 Δ ratio)/M0 Δ ratio).

### Hue Feature and CIELab

The red-green-blue (RGB) values were collected from each postoperative scar photographic image for both the scar and the skin regions. The RGB color space has rapid calculation and does not need to calculate coordinate conversions. The CIELab is a color space modeled on the human vision and designed to be perceptually uniform in human perception. Coordinates of CIELab are L, a, and b, respectively, where L* represents the lightness of the color (L*=0 indicates black and L*=100 indicates diffuse white), a* represents the position between red and green (negative values means green and positive values means red) and b* represents the position between yellow and blue (negative values means blue and positive values means yellow). Conversion between RGB and CIELab is a two-stage process according to the equations of the previous study (Kryjak et al., J Real Time Image Process, 2014).

### Textual Features

Skin texture, the perception of roughness and directionality of human vision, has a significant effect on the scar cosmesis which can be quantified and calculated. The Gray-level Co-occurrence Matrix (GLCM) describes properties about the spatial distribution of the gray levels between adjacent pixels in an image. This study utilized second-order statistics based on the GLCM to quantify the skin variation induced by scarring, including 4 different eigenvalues: contrast, homogeneity, correlation, and entropy. Normalization was performed before the GLCM eigenvalue extracted and the sum of the elements of GLCM was set as 1 for computing. The eigenvalues used are discussed below:

### Contrast (Con)

This eigenvalue is used to measure the amount of the local grey level variations in an image, which is the intensity contrast between adjacent pixels in each region. A larger gray level difference represents a larger Con value of GLCM.

### Homogeneity (Hom)

This reflects the similarity of image textures and scaled the local changes of image texture. High values of homogeneity denote the absence of intra-regional changes and locally uniform grayness of the image.

### Correlation (Cor)

The correlation is used to measure the grey level linear dependencies in an image. For example, when the number of the textures in the horizontal direction is more than other directions, the value of the correlation feature is higher along this direction.

### Entropy (ENT)

This term is originated from thermodynamics and reflects the non-uniformity, randomness of greyscale distribution in image texture processing. The more scattered the greyscale distribution is, the higher of the entropy values.

### Statistical Analysis

Differences between the results derived from the topical formulation-treated side and the control side were analyzed with independent t-test with SPSS version 22.0 (SPSS Inc., Chicago, IL). Statistical significance was accepted at p<0.05.

### Result

A total number of 46 human patients (female 57%, mean age 52 years old) were subject to this study. Table 1 shows the overall distribution and characteristics of all the participating patients.

TABLE 1
Demographic Information and the assessed scar sites

| Characteristics | |
|---|---|
| Gender | |
| Male | 20 |
| Female | 26 |
| Mean age | 51.71739 (20-92) |
| Operation | |
| Thyroidectomy | 31 |
| Parathyroidectomy | 1 |
| Neck disscetion | 2 |
| Excision of Submandibular gland | 2 |
| Parotidectomy | 3 |
| Excision of Neck mass | 4 |
| Inguinal hernia repair | 3 |
| AST | 18.2973 (5-36) |
| ALT | 15.7027 (4-51) |
| Creatinine | 1.5 (0.4-5.4) |

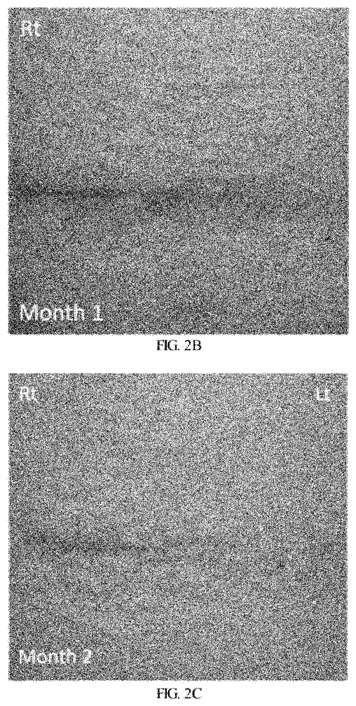

No side effects such as maceration or infection were observed. All patients reported that both creams were easy to apply. 67% of the patients did not have post-operative scar itch or pain. Fifteen patients reported mild itching on the right side and seven of them also described itching on the left side. An excellent response to the topical formulation treatment was observed in all cases, including reduction in redness, and texture improvement. Exemplary results are shown in FIGS. 2A-2D.

At the end of the study, a clinical assessment using either VSS or mVSS was performed on each of the patient, and all parameters showed a significantly lower value (indicating scar improvement) on the side treated with the topical formulation relative to the placebo cream, including significant differences in pigmentation, vascularity, pliability, height and itchiness. Patients also rated their overall satisfaction, with results on the topical formulation-treated side showing a higher VAS with a significant difference and relatively modest effect on QoL. The results are summarized in Table 2 below.

TABLE 2
Subjective scoring of scar quality.

| Subjective assessment, Mean (SD) | Lt, Fespixon | Rt, control | p-value |
|---|---|---|---|
| VAS from patient (0-10 points) | | | |
| Satisfaction | 8.43 (1.54) | 7.56 (1.72) | 0.012 |
| QoL | 1.17 (1.58) | 1.61 (1.68) | 0.205 |

VSS from doctors & mVSS from PI

| | | | |
|---|---|---|---|
| Pigmentation (0-3 points) | 1.21 (0.79) | 1.57 (0.83) | 0.043 |
| Vascularity (0-3 points) | 0.92 (0.77) | 1.34 (0.73) | 0.013 |
| Pliability (0-5 points) | 0.58 (0.69) | 0.95 (0.81) | 0.026 |
| Height (0-3 points) | 0.36 (0.40) | 0.66 (0.47) | 0.002 |
| Pain (0-3 points) | 0.02 (0.15) | 0.12 (0.39) | 0.150 |
| Itchiness (0-3 points) | 0.16 (0.37) | 0.37 (0.54) | 0.039 |

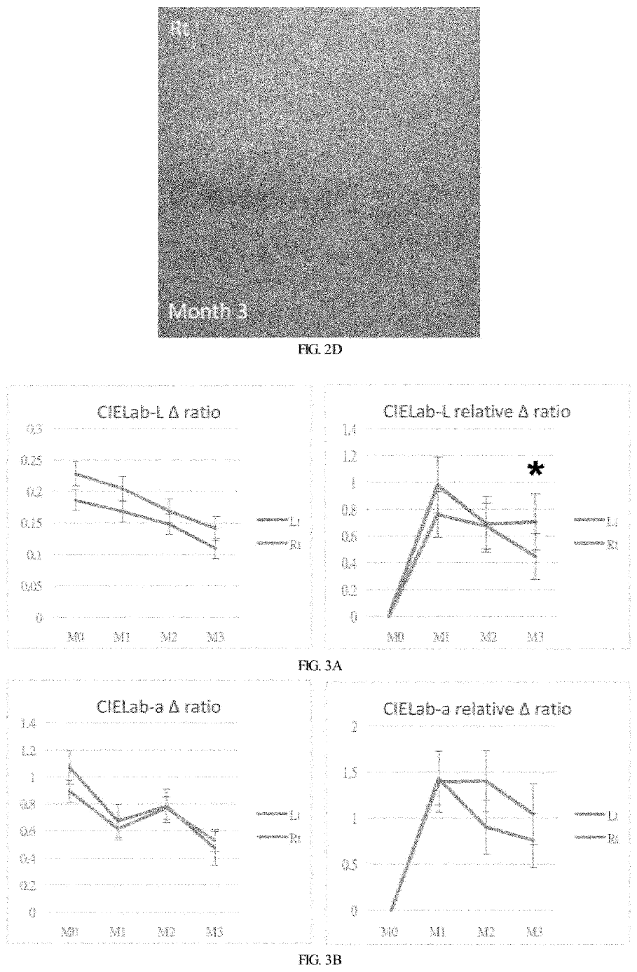

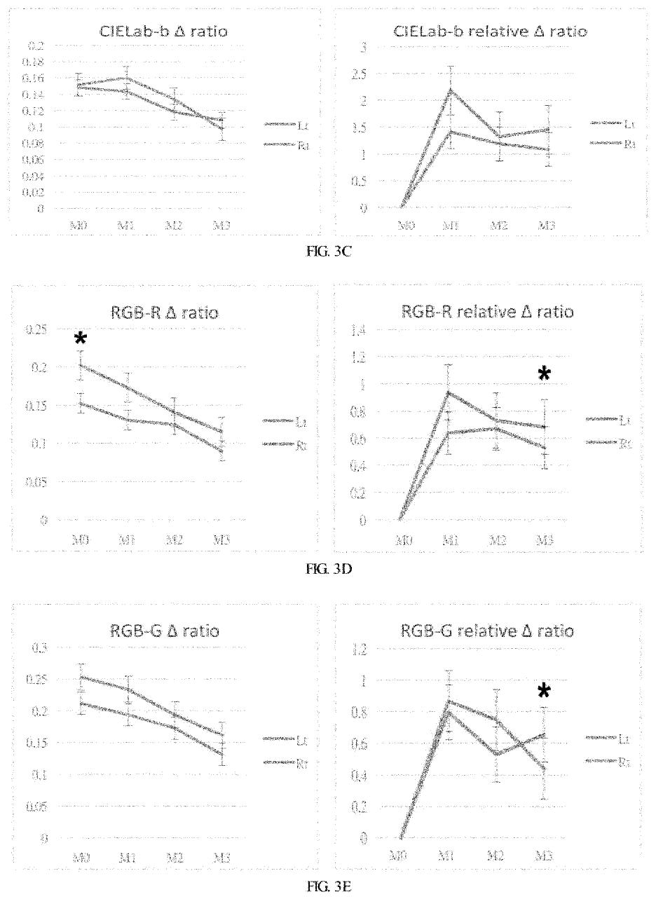

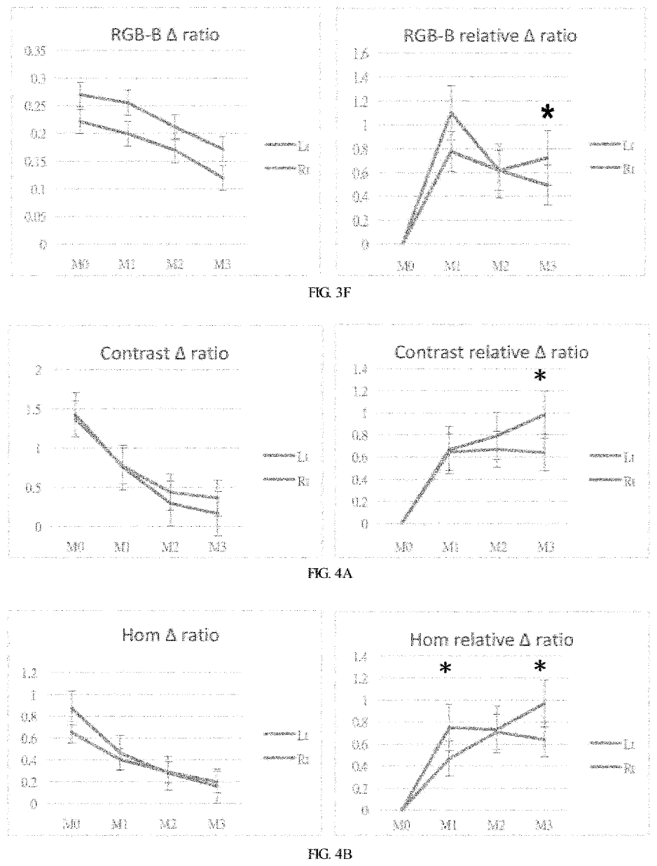

The results of the quantitative analysis of texture showed a decreasing and increasing trend in the difference ratio and relative difference ratio, respectively. See Table 3 below. These results coincide with the maturation process of the scars, indicating that the quantitative analysis by the digital photographs is an ideal tool with discriminatory power. Quantitative analysis of the color includes eigenvalues from the CIELab and RGB systems, calculating differences and relative differences ratio and graphing the trends (FIGS. 3A-3F). The results demonstrated a significant improvement in relative difference ratio of colour lightness (“L” of the CIELab) on the topical formulation-treated side at month 3 (p<0.0001), suggesting less hyperpigmentation than the control side. There were notable trends towards increased relative difference ratios on the Fespixon treated side by the end of this study, with significant differences in all RGB trends (R, p=0.034; G, p=0.001; B, p=0.011), suggesting relatively accelerated scar maturation compared to the control side, as the immature scars looked redder.

TABLE 3
Trends in color and texture difference ratios and relative difference ratios.

| | | | | | Relative difference ratio: | | | |
|---|---|---|---|---|---|---|---|---|
| | Difference ratio: | | | | relative Δ ratio = (Δ ratio of Mx − | | | |
| | Δ ratio = Δ(scar − skin)/skin | | | | Δ ratio of M 0)/A ratio of M 0 | | | |
| | M 0 | M 1 | M 2 | M 3 | M 0 | M 1 | M 2 | M3 |
| CIELab - L | 0.206996 | 0.186226 | 0.15831 | 0.125118 | 0 | 0.869186 | 0.680006 | 0.57634 |
| CIELab - A | 0.981732 | 0.643906 | 0.776719 | 0.500905 | 0 | 1.413148 | 1.151311 | 0.901146 |
| CIELab - B | 0.14984 | 0.151838 | 0.125897 | 0.102589 | 0 | 1.797213 | 1.25486 | 1.266351 |
| Hue - R | 0.177208 | 0.151671 | 0.132636 | 0.10264 | 0 | 0.788043 | 0.699152 | 0.60582 |
| Hue - G | 0.23196 | 0.213646 | 0.182837 | 0.14616 | 0 | 0.83073 | 0.637373 | 0.545833 |
| Hue - B | 0.245763 | 0.227621 | 0.190235 | 0.145948 | 0 | 0.938681 | 0.616196 | 0.607824 |
| Contrast | 1.398777 | 0.775644 | 0.366085 | 0.262526 | 0 | 0.669215 | 0.730696 | 0.812308 |
| Homogeneity | 0.763749 | 0.420933 | 0.284099 | 0.17821 | 0 | 0.558188 | 0.72146 | 0.80463 |
| Correlation | 0.052052 | 0.035889 | 0.02085 | 0.019019 | 0 | 0.54906 | 0.72411 | 0.893672 |
| Entropy | 0.132379 | 0.088022 | 0.072313 | 0.047838 | 0 | 0.385284 | 0.717647 | 0.74886 |

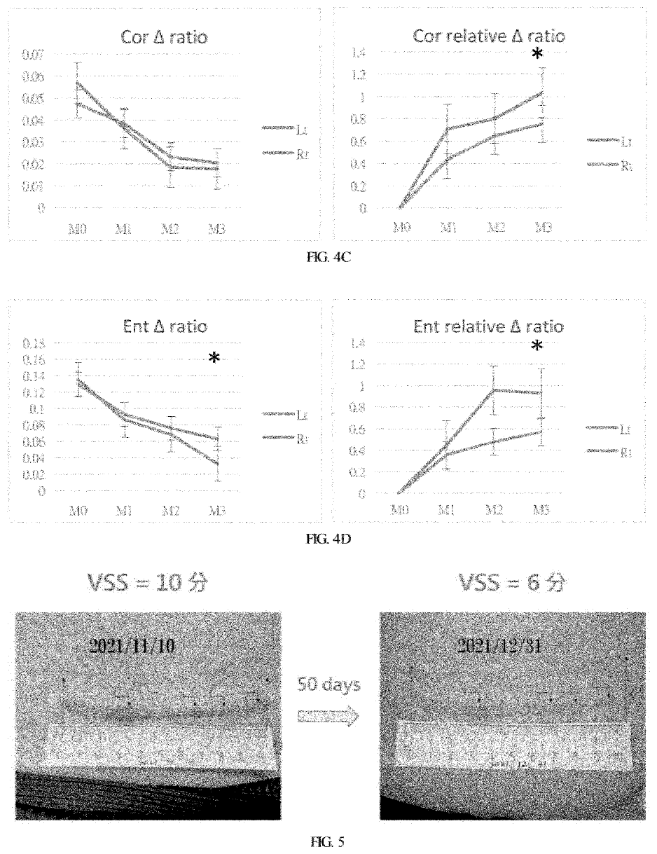

Quantification of the texture eigenvalues using Con, Hom, Cor, Ent of GLCM to derive differences and relative differences ratio and graphs of trends was performed and the results are shown in FIGS. 4A-4D.

The outcomes revealed that at month 3, the difference ratio of Ent was significantly decreased on the topical formulation-treated side (p=0.010), indicating less disorganization and more similar to unbroken intact skin as compared to the control side. At the end of this study, there were statistically significant improvements of relative difference ratio in all texture eigenvalues on the topical formulation-treated side (Con, p<0.0001; Hom, p<0.0001; Cor, p=0.011; Ent, p<0.0001), reflecting increased consistency and gray level linearity with reduced complexities compared to the control side. The trend of color and texture showed increased relative difference ratio with significant difference in most of the eigenvalues toward topical formulation-treated side, which corresponded with relatively rapid scar maturation.

Overall, the results reported herein suggested the analysis could be discriminative as well the outstanding validation of the topical formulation in scar cosmesis.

### Example 3: Case Studies for Use of Topical Formulation to Promote Scar Maturation

This example provides a case study showing that the topical formulation disclosed herein successfully promoted scar maturation in human patients.

A female patient aged 29 has post-surgical (caesarean section) hypertrophic scar for 1 year. This patient was subject to the treatment with the topical formulation disclosed herein (see Example 2 above). Briefly, the topical formulation was applied to the scar areas. Scar assessment was preformed before the treatment and on Day 50 post-treatment and the results are shown in Table 4 below. See also FIG. 5.

TABLE 4
VSS Assessment Before and After Treatment

| | VSS | | | | |
|---|---|---|---|---|---|
| | Value | Pliability | Height | Vascularity | Pigmentation |
| Before | 10 | 3 | 2 | 3 | 2 |
| treatment | | | | | |
| Day 50 | 6 | 2 | 1 | 2 | 1 |
| post | | | | | |
| treatment | | | | | |

### Other Embodiments

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions. Thus, other embodiments are also within the claims.

### EQUIVALENTS

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

1. A method for promoting maturation of a scar or reducing the risk of scar formation in a subject, the method comprising: administering to a subject at a skin site in need thereof an effective amount of a topical formulation, which comprises a Plectranthus Amboinicus (PA) extract, and one or more excipients.

2. The method of claim 1, wherein the PA extract comprises salvigenin, cirsimaritin, rosmarinic acid, carvacrol, or a combination thereof.

3. The method of claim 1, wherein the PA extract is prepared by a process comprising:
(i) mixing a part of PA with an extracting solution to produce a first PA extract,
(ii) filtrating and concentrating the first PA extract to produce a concentrated PA extract;
(iii) contacting the concentrated PA extract onto a hydrophobic interaction chromatography resin, and
(iv) eluting the column with an eluent solution to product the PA extract.

4. The method of claim 3, wherein the part of PA in step (i) is an above-ground part.

5. The method of claim 3, wherein the extracting solution is acetone, butyl methyl ether, ethanol, ethyl acetate, isopropyl alcohol, methanol, or a mixture thereof.

6. The method of claim 3, wherein the eluent solution comprises a solvent having a polarity index of about 2.1-5.4.

7. The method of claim 3, wherein the eluent solution comprises a mixture of at least two solvents selected from the group consisting of acetone, ethanol, ethyl acetate, and hexane.

8. The method of claim 1, wherein the topical formulation further comprises a Centella asiatica (CA) extract.

9. The method of claim 8, wherein the CA extract comprises asiaticoside.

10. The method of claim 8, wherein the topical formulation comprises the PA extract and the CA extract at a weight ratio of 1:1 to 1:4; optionally 1:4.

11. The method of claim 10, wherein the topical formulation comprises about 0.25% (w/w) of the PA extract and/or about 1% (w/w) of the CA extract.

12. The method of claim 1, wherein the one or more excipients in the topical formulation comprise: (a) a viscosity increasing agent, (b) an ointment base, (c) an antimicrobial agent, and (d) an emulsifying agent.

13. The method of claim 12, wherein, in the topical formulation:
the viscosity increasing agent is about 1.0-10% (w/w),
the ointment base is about 5-30% (w/w),
the antimicrobial agent is about 0.01-0.2% (w/w), and/or
the emulsifying agent is about 0.5-6% (w/w).

14. The method of claim 13, wherein:
the viscosity increasing agent comprises cetostearyl alcohol, cholesterol, stearyl alcohol, chlorocresol, white wax, stearic acid, cetyl alcohol, or a combination thereof, optionally wherein the viscosity increasing agent comprises cetostearyl alcohol;
the ointment base comprises one or more petrolatum compounds, optionally liquid petrolatum and white petrolatum;
the antimicrobial agent comprises one or more paraben compounds, optionally methyl paraben and propyl paraben; and/or
the emulsifying agent comprises sortiban and/or polysorbate, optionally sorbitan monostearate and polysorbate 60.

15. The method of claim 12, wherein the one or more excipients further comprise one or more solvents, which optionally are propylene glycol and water.

16. The method of claim 8, wherein the topical formulation comprises the Plectranthus amboinicus extract and the Centella asiatica extract in a total amount of about 0.5-5% (w/w), cetostearyl alcohol in an amount of about 1.0-10% (w/w), a combination of white petrolatum and liquid petrolatum in a total amount of about 5-30% (w/w), a combination of methyl paraben and propyl paraben in a total amount of about 0.01-0.2% (w/w), and a combination of sorbitan monostearate and polysorbate 60 in a total amount of about 0.5-6% (w/w).

17. The method of claim 1, wherein the scar is a post-operative scar or a scar caused by a skin injury.

18. The method of claim 1, wherein the scar is a keloid scar or a hypertrophic scar.

19. The method of claim 1, wherein the subject is a human patient.

20. The method of claim 18, wherein the scar is a post-operative scar, and wherein the subject has undergone or is scheduled for a surgical procedure at the skin site.

21. The method of claim 1, wherein the topical formulation is in a form of cream, gel, dressing, spray formulation, ointment, paste, patch, mask, or lotion, optionally in a form of cream.

22. The method of claim 1, wherein the topical formulation is administered to the skin site 1-4 times per day.

23. The method of claim 1, further comprise assessing skin conditions at the skin site where the topical formulation is applied before, during, and/or after the treatment.

24. The method of claim 23, wherein the skin conditions comprise pigmentation, vascularity, pliability, height of scars, hue feature, textual feature, or a combination thereof.

25. The method of claim 23, wherein the assessing step comprises semi-quantitative scar assessment, quantitative digital photography analysis, or a combination thereof.

26. The method of claim 25, wherein the assessing step comprises the quantitative digital photography analysis, which comprises:
(a) obtaining digital images of one or more scar areas and one or more skin areas nearby the scar areas at the skin site where the topical formulation is applied;
(b) processing the digital images to calculate hue features and textural features of the scar areas and the skin areas; and
(c) comparing the hue features and textual features of the scar areas with those of the skin areas to assess scar maturation.

27. A method for assessing scar maturation during a treatment, the method comprising:
(a) obtaining digital images of one or more scar areas and one or more skin areas nearby the scar areas at a skin site where a treatment is applied before and during the course of the treatment;
(b) processing the digital images to calculate hue features and textural features of the scar areas and the skin areas; and
(c) comparing the hue features and textual features of the scar areas with those of the skin areas to assess scar maturation.
